(19) United States
(12) Patent Application Publication
(10) Pub. No.: US 2024/0099649 A1
(43) Pub. Date: Mar. 28, 2024

(54) METHOD AND SYSTEM FOR SUPPORTING MOTHERS OF NEWBORN INFANTS DURING BREASTFEEDING

(71) Applicant: PICTERUS AS, Trondheim (NO)

(72) Inventors: Tormod THOMSEN, Aalesund (NO); Leonardo RUSPINI, Trondheim (NO); Cynthia MAIER, Delafield, WI (US)

(21) Appl. No.: 17/952,260

(22) Filed: Sep. 25, 2022

(51) Int. Cl. A61B 5/00; A61B 5/145; G16H 40/67

(52) U.S. Cl. CPC A61B 5/4288; A61B 5/0022; A61B 5/14546; A61B 5/4866; A61B 5/7267; A61B 5/7282; A61B 5/7465; G16H 40/67; A61B 2503/045

(57) ABSTRACT

A method and system for remotely supporting breastfeeding mothers by receiving, at a cloud application in communication with at least one instance of mobile device application, breastmilk transfer data from at least one breastfeeding sensor configured to monitor breastmilk transfer from a mother to an infant during a breastfeeding period, and bilirubin data from at least one bilirubin sensor configured to monitor serum bilirubin level in the infant during the breastfeeding period, performing an analysis of the received breastmilk transfer data and the received bilirubin data, and performing at least one action in response to applying predetermined rules to the analysis.

### TECHNICAL FIELD

The present disclosure relates generally to the field of healthcare and more specifically to a new and useful system and automated method for supporting mothers of newborn infants with breastfeeding via remote monitoring and communications.

### BACKGROUND

The benefits of breastfeeding are recognized by medical societies the world over, and significant governmental and non-profit agency resources are invested in encouraging new mothers to breastfeed their newborn infants. The World Health Organization (WHO) states that breastfeeding “is one of the most effective ways to ensure child health and survival.” According to the WHO, breastmilk is “the ideal food for infants. It is safe, clean and contains antibodies which help protect against many common childhood illnesses. Breastmilk provides all the energy and nutrients that the infant needs for the first months of life.”

The American Academy of Pediatrics (AAP) and the Centers for Disease Control and Prevention (CDC) recommend that infants should be exclusively breastfed for the first six months of life and then continue to receive breastmilk alongside table foods for at least the first 1 to 2 years of life. Optimal nutrition during this period lowers morbidity and mortality, reduces the risk of chronic disease, and fosters better development overall. The AAP advises that infants who are breastfed have decreased risk of: otitis media, diarrhea, respiratory tract infection, necrotizing enterocolitis, SIDS, atopic dermatitis, asthma, celiac disease, Crohn's Disease and ulcerative colitis, late-onset sepsis in preterm infants, type 1 and type 2 diabetes, leukemia, and childhood overweight and obesity. In addition, multiple important health benefits for the mother are also associated with breastfeeding. The WHO estimates that 45% of all child deaths are associated with inadequate nutrition and that breastfeeding could save the lives of over 820 000 children under the age of 5 years each year. The WHO recommends early initiation of breastfeeding within 1 hour afterbirth. However, only about 44% of infants aged 0-6 months worldwide were exclusively breastfed over the period of 2015-2020.

Over 98% of infants born in the United States are born in the hospital setting. Mothers receive breastfeeding support while in the hospital from pediatricians, nurses and lactation counsellors. However, mothers and newborns are being released from hospitals significantly earlier than in previous decades so receive less breastfeeding support and education from in-hospital healthcare providers prior to their release than in prior years. In some cases, the mother and infant are released from the hospital sooner than 24 hours after the baby is born. Many mothers experience significant difficulties breastfeeding after they leave the hospital and do not have adequate support at home to encourage them to continue attempting to breastfeed while their crying, hungry infant demands an immediate response. In addition, up to 20% of mothers suffer from postpartum depression, which can make adherence to breastfeeding guidelines much more difficult. Phone calls to nurses or lactation counsellors at the hospital or visits to breastfeeding clinics can help provide the needed support; however, these resources are not available to new mothers in all regions, especially in rural areas. While a simple phone call can often answer questions and clarify breastfeeding instructions that may not have been fully understood, these calls are resource intensive for the hospital and are not always effective due to the lack of visual feedback for the healthcare provider about the effectiveness of latching and milk transfer, as well as the overall appearance and behavior of the infant. Moreover, phone calls require a synchronous communication which relies on the availability of appropriate hospital personnel at the moment the mother is seeking support. Email capabilities, as well as existing patient portals implemented by many hospitals, offer asynchronous communications but fail to provide emotional support or react with immediacy to information about the mother and infant.

The Center for Disease Control and Prevention (CDC) in the United States estimates the number of mothers who have difficulty breastfeeding without hospital support at 1 in 3, and 1 in 3 new mothers stop breastfeeding without it. According to the Surgeon General, nearly 50% of women stop breastfeeding within the first two weeks postpartum because of their apprehension about the sufficiency of breast milk that the infant is receiving. The CDC estimates that these low rates of breastfeeding increase health costs by $2.2 billion per year in the United States.

Multiple device solutions exist for measuring parameters related to an infant's breast milk intake that may be used in the home setting to help monitor whether a newborn infant is receiving sufficient nutrition via breastfeeding. For example, specialized infant scales that accurately measure the infant's weight prior to and after breastfeeding sessions can calculate the amount of milk transferred to the infant. Alternatively, weighing of diapers to calculate the infant's urine output has also been relied on to help assess the success of breastfeeding. Versions of these scales have recently become available for use by parents in the home setting. Some “smart” versions of these infant scales also have the capability to communicate wirelessly with a software tracking application that tracks changes in the infant's weight, amount consumed at feeding sessions, and for some models, the rate of change in the infant's weight. The tracking software may run locally on a mobile device solely for the parents' reference, or it may communicate the measurement results to a remote cloud-based server for access by a healthcare provider. Other technologies for measuring parameters related to breast milk intake include devices like the Momsense® (Momsense Ltd., Ramat Gan, Israel), a patented technology that assesses acoustics as the baby swallows. These acoustics are then converted to trackable feeding patterns which may be analyzed to confirm an appropriate latch and correct swallowing during breastfeeding. The MilkSensem breastfeeding monitor sold by Walmart measures the impedance of breast tissue before and after breastfeeding. The change in measured impedance is claimed to correlate to the quantity of milk transferred to the infant. Molkenthin et al. in US Patent Application Publication US 2022/0022842 describe a system and method for using ultrasound to measure changes in the infant's stomach during breastfeeding to determine the volume of milk that is being transferred to the infant. The Coro breastfeeding monitor sold by Coroflo is a nipple shield that monitors the quantity of milk flowing through it and is paired with a smartphone app that connects to a cloud-based application for analysis and calculation of metrics related to the success of breast-feeding. Shechter Uzhpizin et al. in US Patent Application Publication US 2022/0087594 describe a method and device for determining adequacy of breastmilk by analyzing samples of milk at two different time points to determine changes in its chemical composition. The method determines the success of breastfeeding based on detecting normal/abnormal patterns of changes to the milk's chemical composition as the mother's milk production strengthens in the days after the infant's birth. While these devices all measure different parameters related to breastfeeding, their shared purpose is to assist mothers to determine whether the transfer of milk to their infant is adequate.

In addition to concerns about whether the newborn infant is receiving adequate nutrition, breastfed infants are at higher risk for developing severe levels of bilirubin and jaundice than bottle fed infants. Moreover, insufficient transfer of milk can lead to low urine output, which causes higher levels of bilirubin to build up in the blood instead of being excreted in the urine. While rare, if severe jaundice is not promptly diagnosed and treated, a debilitating type of brain damage called kernicterus can occur. The AAP therefore recommends that mothers of all newborns be provided with educational information about the risks of neonatal jaundice and importance of breastfeeding before they are discharged from the hospital. In addition, AAP recommends that a baseline measurement of bilirubin levels in newborns be performed at the hospital prior to discharge using either Total Serum Bilirubin (TSB) testing of a blood sample or noninvasive Transcutaneous Bilirubin (TCB) measurement. For infants who test at the hospital with a bilirubin level above a threshold value for the infant's gestational age and the age in hours after birth, the AAP recommends treatment using phototherapy along with a prolonged stay in the hospital setting. For infants with low bilirubin levels in the hospital, the risk of subsequently developing dangerous levels of bilirubin during the days after release is low, so an in-person visit to assess the infant's health and success of breastfeeding may be scheduled later during the first week, according to the local availability of healthcare professional resources. Infants whose bilirubin levels are in an intermediate range however, require more aggressive follow-up during the days after release from the hospital, sometimes with daily hospital or clinic visits and blood sampling. The timing and type of follow-up is determined, in part, by the projected risk of the infant's bilirubin levels increasing above the treatment threshold as provided in the AAP guideline and, in part, by the physician's assessment of the mother's ability to breastfeed and provide adequate care for the infant at home.

The gold standard for measurement of neonatal bilirubin levels is serum blood testing to determine Total Serum Bilirubin levels. While blood samples may be obtained in the home setting, this has traditionally been performed by a visiting healthcare professional and not by the parents themselves, and the blood samples are sent to a lab for processing. Recently, several new innovations have been introduced with the goal of allowing parents to easily collect blood samples and perform the analysis themselves to determine the infant's serum bilirubin levels. The BiliStrip system by Bilimetrix, Italy, is a novel point-of-care blood test consisting of a test strip and reader that is intended for home use and requires minimal user training. The BiliPic system by Bilimetrix, USA, is another point-of-care blood test that uses a smartphone's internal camera to take photos of a test strip and is also intended for home use. These approaches are still invasive and are not appropriate for daily measurements, or for making multiple measurements per day. Noninvasive transcutaneous bilirubinometers designed for hospital use are expensive and complicated to use and are not suitable for home-based use by parents. Recently, a mobile application has been introduced on the market by Picterus AS, Norway, that uses a smartphone's built-in camera to take photos of the infant's skin along with a calibration card to correct for variations in camera performance and/or environmental lighting. The photos of the skin and card are analyzed to calculate an estimate of the infant's bilirubin levels. This mobile application has the potential to be used by parents to make periodic measurements of their baby's bilirubin levels at home after release from the hospital, and the smartphone's connectivity allows the measurement results to be uploaded to a cloud-based application or communicated to a healthcare provider for interpretation.

Patient engagement platforms have been created to provide real-time support for patients with long-term healthcare issues such as mental health issues, diabetes, asthma, and other chronic conditions, as well as for mothers during pregnancy. Many of these platforms support the use of remote patient monitoring devices that can upload measured patient data directly to the platform for later review and evaluation by a healthcare professional, or as a first step, for processing by a computer algorithm to identify situations that require more immediate attention by a healthcare professional. Some of these platforms include educational features for patients and some may serve to remind the patient to take their medication at a certain time while providing encouragement to the patient adhere to a treatment program. Some of these platforms are available as downloadable apps for mobile devices, while others are cloud-based. Some of these platforms may use text messages to communicate with patients, including some based on natural language processing algorithms (i.e., “chatbots”) that can respond to a patients' texts using appropriate and encouraging language in a conversational format. These chatbots may provide appropriate information in response to patients' questions and can identify when a patient should be connected immediately to a healthcare professional for attention. These platforms have shown success in encouraging patient adherence to medical regimens in the home setting and have generally been well accepted by both patients and healthcare professionals, while also reducing the cost of care.

### SUMMARY

The present disclosure relates generally to the field of healthcare and more specifically to a new and useful system and automated method for supporting mothers of newborn infants with breastfeeding via remote monitoring and communications. In light of the shortcomings of known methods and systems, there is a need for a generally affordable and accessible approach for healthcare providers to remotely support new mothers with breastfeeding during their first days at home with their babies. A new and useful system and automated method is disclosed that may provide mothers with emotionally responsive messaging and appropriate educational content, as well as monitoring breastfeeding progress and the baby's health and alerting a healthcare professional when their attention and direct communication with the mother is needed. The subject technology extends the supportive interaction of healthcare providers with new mothers during the days after the mother/infant pair leave the hospital by complementing live communications with chatbot interactions and remote measurements of key parameters related to breastfeeding success and infant health. Further, from a public health perspective, mother/infant pair datasets stored during use of the technology, when analyzed and aggregated, may help improve the understanding of correlations between new mothers' digital communication behaviors, health states, and appropriate interventions to improve adherence to breastfeeding guidelines.

According to a broad aspect, an automated method for remotely supporting breastfeeding mothers comprises receiving, at a cloud application in communication with a mobile device application, breastmilk transfer data from at least one breastfeeding sensor configured to monitor breastmilk transfer from a mother to an infant during a breastfeeding period; receiving, at the cloud application, bilirubin data from at least one bilirubin sensor configured to monitor serum bilirubin level in the infant during the breastfeeding period; performing an analysis of the received breastmilk transfer data and the received bilirubin data; and performing at least one action in response to applying predetermined rules to the analysis.

In some examples, the method further comprises receiving, at the cloud application, communications provided at the mobile device application by the mother during the breastfeeding period, wherein the communications include received information comprising one or more of: information about an emotional or physical state of the mother, information about comfort of the mother with breastfeeding, and an assessment by the mother of a physiological state of the infant, and performing the analysis comprises analyzing the received information.

In some examples, the at least one action comprises creating an event flag in association with the mother to trigger attention from at least one healthcare provider.

In some examples, creating the event flag comprises one or more of: displaying a visual indicator on a display associated with the cloud application and initiating a digital communication between the cloud application and the at least one healthcare provider.

In some examples, the at least one action comprises: refraining from creating an event flag in association with mother; and monitoring for one or more of (i) subsequent breastmilk transfer data from the at least one breastfeeding sensor; (ii) subsequent bilirubin data from the at least one bilirubin sensor; and (iii) subsequent communications provided at the mobile device application.

In some examples, the analysis comprises comparing the received breastmilk transfer data and the received bilirubin data to predetermined threshold values and/or to each other.

In some examples, the analysis comprises calculating an individualized risk factor associated with the mother and/or the infant using the received information, the received breastmilk transfer data, and the received bilirubin data.

In some examples, the individualized risk factor is calculated using one or more of: clinical information associated with the mother and/or the infant entered by at least one healthcare provider; and a neural network model trained with reference data.

In some examples, the at least one action comprises providing individualized content to the mother via the mobile device application based on the individualized risk factor.

In some examples, the communications provided by the mother comprise text communications and the method further comprises extracting the received information using a natural language processing algorithm configured to parse the text communications for key words and/or phrases associated with stress and/or health of the mother and/or the infant.

In some examples, the method further comprises receiving, at the cloud application, additional communications provided at additional instances of the mobile device application by additional mothers of additional infants, the additional communications including additional received information; receiving, at the cloud application, additional breastmilk transfer data from additional breastfeeding sensors configured to monitor breastmilk transfer from the additional mothers; receiving, at the cloud application, additional bilirubin data from additional bilirubin sensors configured to monitor serum bilirubin level in the additional infants; performing additional analyses of the additional information, the additional breastmilk transfer data, and the additional bilirubin data; and performing additional actions in response to applying the predetermined rules to the additional analyses.

### BRIEF DESCRIPTION OF THE DRAWINGS

FIG. 1 is a schematic drawing illustrating an exemplary system for supporting breastfeeding in mothers of newborn babies via remote monitoring and communications in accordance with aspects of the present disclosure;

FIG. 2 is a schematic drawing illustrating another exemplary system for supporting breastfeeding in mothers of newborn babies via remote monitoring and communications in accordance with aspects of the present disclosure;

FIG. 3 is a schematic drawing illustrating another exemplary system for supporting breastfeeding in mothers of newborn babies via remote monitoring and communications in accordance with aspects of the present disclosure;

FIG. 4 is a schematic drawing illustrating another exemplary system for supporting breastfeeding in mothers of newborn babies via remote monitoring and communications in accordance with aspects of the present disclosure;

FIG. 5 is a flow chart illustrating an example of an exemplary automated method for supporting breastfeeding in mothers of newborn babies via remote monitoring and communications in accordance with aspects of the present disclosure;

FIG. 6 illustrates an example of an automated communication with a mother in accordance with aspects of the present disclosure; and

FIG. 7 illustrates another example of an automated communication with a mother in accordance with aspects of the present disclosure.

### DETAILED DESCRIPTION

The present disclosure describes a system and method for supporting breastfeeding by mothers of newborn infants based on remote monitoring of at least one measure of breastfeeding success, remote monitoring of bilirubin levels in the newborn infant, and, optionally, automated communications with the mother that are based, in part, on the remote monitoring output. A benefit of the subject technology is to provide on-demand support for mothers to continue breastfeeding without the need for the immediate availability of a healthcare provider. This may be accomplished by automating individualized communications to the mother with messages of encouragement and relevant feedback that are based in part on the remote monitoring output and includes automated identification of situations when real-time support from a healthcare provider is desirable or necessary.

The detailed description set forth below describes various configurations of the subject technology and is not intended to represent the only configurations in which the subject technology may be practiced. The detailed description includes specific illustrative examples for the purpose of providing a thorough understanding of the subject technology. Accordingly, specific details presented in the exemplary systems and methods are provided as non-limiting examples. It will be apparent to those skilled in the art that the subject technology may be practiced without these specific details. In some instances, well-known structures and components are shown in block diagram form to avoid obscuring the concepts of the subject technology.

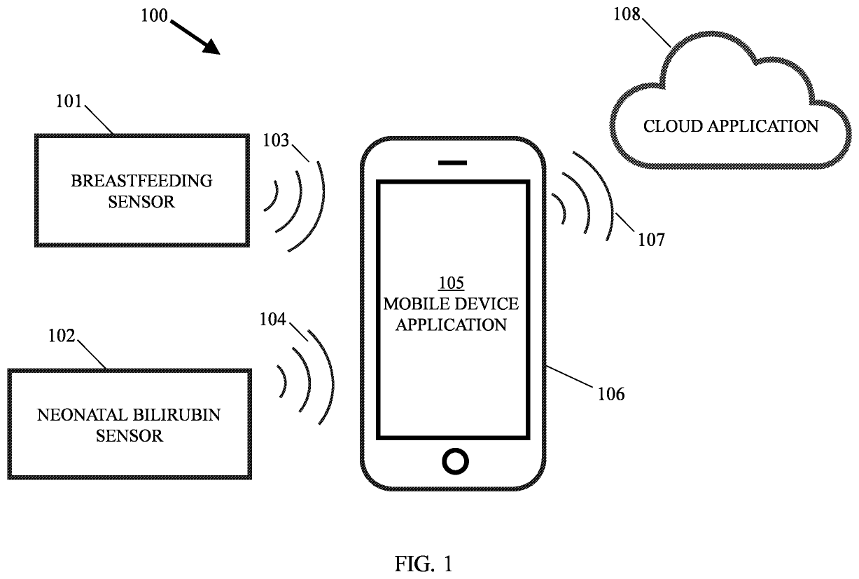

FIG. 1 illustrates a system 100 in accordance with various examples of the present disclosure for supporting breastfeeding by mothers of newborns. The system 100 may include a Breastfeeding Sensor 101 that is configured to monitor breastfeeding by collecting data based on an aspect of breastfeeding or the infant's physiology that is correlated with the transfer of breastmilk from the mother to the infant during a breastfeeding period (i.e. “breastmilk transfer data”). The system 100 may also include a Neonatal Bilirubin Sensor 102 that is configured to monitor the infant's bilirubin levels by collecting data based on the infant's physiology that is correlated with serum bilirubin levels, (i.e. “bilirubin data”). The Breastfeeding Sensor 101 is configured to communicate via a communication channel 103 with a Mobile Device Application 105 that is running on a Mobile Device 106, while the Neonatal Bilirubin Sensor 102 is configured communicate via another communication channel 104 with the Mobile Device Application 105. In some examples, communications over the communication channels 103 and 104 may comprise short-range wireless communications such as Bluetooth® or WiFi communications. In other examples, the Sensors 101 and 102 may communicate with the Mobile Device Application 105 using wired connections, or a combination of wired and wireless connections. The Mobile Device 106 may comprise, for example, a smartphone, tablet, laptop, or other specialized device designed to support the disclosed technology. The Sensors 101 and 102 may be configured to communicate respective measurement outputs (i.e., breastmilk transfer data and bilirubin data) to Mobile Device Application 105 via the respective communication channels 103 and 104.

The Mobile Device Application 105 is configured to communicate via a communication channel 107 with a Cloud Application 108 running on computer hardware remote from the Mobile Device 106 and the mother/infant pair. In some examples, communications over the communication channel 107 may comprise long-range wireless communications, such as cellular communications or internet communications. In other examples, the communication channel 107 may be based on wired connections, or a combination of wired and wireless connections. The Mobile Device Application 105 may be configured to communicate the breastmilk transfer data and/or the bilirubin data to the Cloud Application 108 via the communication channel 107. In some examples, the Cloud Application 108 may be configured to analyze the breastmilk transfer data and/or the bilirubin data. In other examples, the Mobile Device Application 105 may be configured to perform an analysis of the breastmilk transfer data and/or the bilirubin data first and then communicate the results of the analysis to the Cloud Application 108. In some examples, the measurement outputs of the Sensors 101 and 102 may be displayed to the mother, for example, via the Mobile Device Application 105, in addition to being displayed via the Cloud Application 108 to the remote healthcare providers. In other examples, the measurement outputs may be displayed only via the Cloud Application 108 to the remote healthcare providers in order to avoid possible misinterpretation by the mother.

In some examples, the Breastfeeding Sensor 101 may comprise a weighing scale for weighing the infant before/after breastfeeding, or for tracking the infant's weight gain over days, or for tracking the velocity of the infant's weight gain over days. In other examples, the Breastfeeding Sensor 101 may comprise a scale for weighing the infant's diapers. In other examples, the Breastfeeding Sensor 101 may comprise a device that measures changes in characteristics of the breast before and after breastfeeding, including but not limited to impedance-based devices. In other examples, the Breastfeeding Sensor 101 may comprise a device such as an ultrasound-based device that measures flow through the nipple, or it may comprise a nipple guard that directly measures flow through itself to calculate the transfer of milk to the infant. In some examples, the Breastfeeding Sensor 101 may comprise part of the Mobile Device Application 105 and may estimate the milk intake based on artificial intelligence (AI) processing of an acoustic signal received by a microphone of the Mobile Device 106. Those who are skilled in the art will recognize that other possible technologies may be employed to measure a parameter that is related to breastfeeding success and thereby generate the breastmilk transfer data. In some examples, the Mobile Device Application 105 may be configured to analyze data from the Breastfeeding Sensor 101 and to calculate an estimate of transferred breastmilk in mL or other suitable unit.

In some examples, the Neonatal Bilirubin Sensor 102 is configured to measure a parameter related to neonatal serum bilirubin levels based on visual light characteristics of the infant's skin or the sclera of the eyes, for example, reflectance characteristics for visual wavelengths. In some examples, the Neonatal Bilirubin Sensor 102 may comprise a Transcutaneous Bilirubinometer that is adapted for home use. In some examples, the Neonatal Bilirubin Sensor 102 may comprise a spectrophotometer that is adapted for home use by parents to measure reflectance of neonatal skin or sclera and calculate an estimate of serum bilirubin levels. In other examples, the Neonatal Bilirubin Sensor 102 may comprise a blood test kit adapted for home use by parents, with digital output and connectivity with the Mobile Device 106. In one example, the blood test kit may be configured to transfer bilirubin data to the Mobile Device Application 105 via the communication channel 104. In another example, results generated by the blood test kit may be manually entered by the mother into the Mobile Device Application 105, for example, using a keyboard of the Mobile Device 106. Those who are skilled in the art will recognize that other possible technologies may be employed to measure a parameter that is related to serum bilirubin levels and thereby generate the bilirubin data. In some examples, the Mobile Device Application 105 may be configured to analyze data from the Neonatal Bilirubin Sensor 102 and calculate an estimate of serum bilirubin levels.

The Cloud Application 108 may provide an informational and communications interface for remote healthcare providers who are responsible for monitoring the mother/infant pair during the breastfeeding period and may display the breastmilk transfer data and the bilirubin data to the healthcare providers for their review and analysis via an appropriately designed user interface that is remote to the mother/infant pair. For example, the Cloud Application 108 may include web application functionality that allows a healthcare provider to access information about the mother/infant pair, including the breastmilk transfer data and the bilirubin data, from their choice of computer or mobile device. The Cloud Application 108 may include functionality for analyzing the breastmilk transfer data and the bilirubin data and for displaying metrics to the healthcare provider related to the degree of breastfeeding success and/or the infant's risk for developing higher levels of bilirubin that may require a therapeutic intervention. The Cloud Application 108 may continually update the displayed information for the healthcare provider as new breastmilk transfer data and new bilirubin data is received. The Cloud Application 108 may include functionality for analyzing the breastmilk transfer data and the bilirubin data, for identifying concerning values of the data, and for automatically alerting a healthcare provider that the mother/infant pair needs their attention, for example, via text messages or automated phone calls to the healthcare provider, alerts communicated via an EHR system or other types of alerts.

On receiving the breastmilk transfer data and/or the bilirubin data, the Cloud Application 108 may analyze the received data and automatically generate appropriate content for individualized communications to the mother. The Cloud Application 108 may send the individualized communications to the Mobile Device 106 to be received by the mother, for example, as text messages, voice messages or emails. The individualized communications may be received at the Mobile Device 106 over the communication channel 107, or over some other communication channel between the Cloud Application 108 and the Mobile Device 106. The Cloud Application 108 may also include programmed functionality to receive communications, for example, text messages, voice messages, or emails from the mother sent via the Mobile Device 106. In other examples, the Cloud Application 108 may send the communications to the Mobile Device Application 105 to display the communications and the Mobile Device Application 105 may be programmed to receive communications from the mother and send these to the Cloud Application 108. The communications from the mother may be received at the Cloud Application 108 over the communication channel 107, or over some other communication channel between the Cloud Application 108 and the Mobile Device 106.

The communications sent by the Cloud Application 108 to the Mobile Device 106 may include content that is designed to engage the mother during the breastfeeding period to provide emotional support, to solicit information from the mother to help identify any breastfeeding concerns or issues, to provide relevant educational content when appropriate, to remind the mother to take measurements with the Sensors 101 and/or 102 or to perform other scheduled actions, to respond to the mother's questions, and/or to solicit other information from the mother that may help identify situations when it is desirable or necessary for the mother to receive attention from a healthcare professional.

The Cloud Application 108 may incorporate natural language processing capabilities, based on AI modules, for providing chatbot functionality (i.e., “the chatbot”) that is able to mimic a conversation with a human, providing the mother with emotionally supportive feedback and responses, via, for example, text messages sent to the Mobile Device 106. Patient engagement platforms have shown more benefit when the educational information presented to the patient is personalized and insightful to the individual patient rather than being generalized. The AI chatbot may trained (i.e., using reference data) to identify breastfeeding issues by analyzing the mother's communications and providing individualized educational content to the mother to help resolve the issues. The AI chatbot may be trained to provide educational content to the mother that is selected on the basis of the breastmilk transfer data and/or the bilirubin data and/or information communicated by the mother via the Mobile Device 106. The educational content may be provided to the mother, for example, as text messages with relevant information and/or suggested actions that may be taken by the mother, or in another example, as a text message containing a hyperlink to relevant informational internet video content. The AI chatbot may be trained to recognize a request from the mother to be connected immediately to a healthcare provider or to be placed in a queue for their attention and may be trained to act on the request by performing the requested action. The AI chatbot may be trained to identify indicators of high levels of maternal stress in the mother's responses by parsing her responses for words or phrases that are associated with postpartum depression and/or to screen for other indicators in her responses, for example, concerning information about the infant's physiology, or that a raised level of risk exists for the mother/infant pair. In response to identifying a raised level of concern, the AI chatbot may be trained to flag the attention of a healthcare provider to the mother/infant pair by, for example, sending the healthcare provider a text message, or placing the mother/infant's identifying information in a message queue that is constantly being monitored by a healthcare professional. The healthcare professional may then communicate directly with the mother to provide support either by taking control of the text message conversation from the chatbot or by using other external means, for example, phoning the mother directly, and the healthcare professional may then take any other necessary additional actions to ensure the health of the infant and mother. The chatbot may include voice conversation features via text-to-voice and automated speech recognition functionality that allow audible conversations with the mother via the Mobile Device 106. The chatbot may include functionality for analyzing the mother's speech and voice patterns for indications of stress.

The Cloud Application 108 may be designed to monitor a plurality of mother/infant pairs simultaneously. The Cloud Application 108 may include web application functionality that allows a healthcare provider to access and view a list of mother/infant pairs that includes visual indicators to denote certain information pertaining to the mother/infant pairs. Such information may include, for example, a priority indicator for indicating that direct communication with a healthcare provider is desirable or necessary, a clock display to indicate the time elapsed since the last direct communication with a healthcare provider, an icon to indicate that breastmilk transfer data and/or bilirubin data had been received that were outside a predetermined healthy range of values. The Cloud Application 108 may provide a plurality of healthcare providers with access to the data associated with one or more mother/infant pairs belonging to the plurality of mother/infant pairs. In some examples, the Cloud Application 108 may provide “hospital at home” functionality, including providing infrastructure support for a command center staffed by healthcare providers who monitor many patients at once, including patients with other clinical conditions and monitoring needs. In other examples, the Cloud Application 108 may be specialized for providing support only to new mothers, with restricted access to specialty healthcare providers such as lactation counsellors, nurses working in the newborn infant nursery, and pediatricians.

In some examples, the Cloud Application 108 may run in a public cloud on server hardware resources located in a remote data center, wherein the hardware resources are partitioned among many separate users, for example Amazon Web Services. In some examples, the Cloud Application 108 may run in a hospital's private cloud on hospital-owned or leased dedicated server hardware. The server hardware may be located remotely from the hospital in a data center or may be on dedicated server hardware that is located in the hospital itself. The Cloud Application 108 may be integrated with the hospital's Electronic Health Records (EHR) system, allowing healthcare providers to access the mother's and infant's medical records and/or to leverage patient communication tools that are also integrated with the EHR system for conducting virtual visits with the mother and infant. The Cloud Application 108 may be integrated into the EHR system itself, along with a wide range of other functionality. Those who are skilled in the art will recognize that other possible configurations of software and computing hardware are possible.

The Cloud Application 108 may include functionality for automatically calculating an infant's updated risk of receiving insufficient nutrition or developing clinically relevant bilirubin levels after receiving a communication comprising any one or more of: new measurement outputs from the Breastfeeding Sensor 101, new measurement outputs from the Neonatal Bilirubin Sensor 102, and new information communicated by the mother. The infant's updated risk may be increased as a result of a higher measured bilirubin level or a high rate of increase in the latest measured bilirubin levels; however, the increased risk from the higher or increasing bilirubin levels may be offset by higher volumes of breast milk transfer, which serve to help break down bilirubin in the infant to harmless by-products. The infant's risk for developing actionable levels of bilirubin reflects a balance between breastfeeding success and the current bilirubin level. Even in the absence of high bilirubin levels, however, the infant's overall health risk may be high if breastmilk transfer is insufficient due to inadequate nutrition and weight gain. The Cloud Application 108 may be configured to calculate the infant's overall health risk according to one or more of: a look-up table programmed in or stored in memory; a mathematical equation or correlation derived from reference data; a neural network model trained with reference data and customized based on the specific data already obtained from the mother/infant pair. In some examples, the method of calculating the infant's risk may include referencing a large database of relevant infant data and known outcomes. In some examples, the Cloud Application 108 is configured to calculate risk for the mother/infant pair by including factors related to the mother's health, for example, by parsing the mother's responses for indicators of postpartum depression. The Cloud Application 108 may be programmed to automatically alert a healthcare provider that the mother/infant pair needs their attention via text messages or automated phone calls to the healthcare provider, or other type of alert, when the infant's overall health risk or risk of developing actionable levels of bilirubin exceeds a threshold value.

In some examples, prior to and/or after the mother/infant pair are released from the hospital, healthcare providers may enter clinical data specific to the mother/infant pair into the Cloud Application 108, customizing the behavior of the Cloud Application 108 to provide individualized feedback to the mother and scheduling actions for the mother to perform with appropriate timing. For example, a healthcare provider may enter the results of in-hospital bilirubin testing for a particular infant into the Cloud Application 108. Based on the in-hospital bilirubin levels and the age of the infant at the time of the in-hospital bilirubin measurement was performed, the Cloud Application 108 may suggest a testing schedule for home-based bilirubin measurements using the Neonatal Bilirubin Sensor 102, which the healthcare provider may then either accept or modify according to their professional judgment. Such a testing schedule is a non-limiting example of a set of rules governing message content and timing for communications with the mother that are predetermined before use of the system 100 via a combination of the Cloud Application 108 programming and input entered by the healthcare provider prior to release from the hospital. Such rules may be based on clinical guidelines, for example, the AAP's Clinical Guideline on Hyperbilirubinemia or other such jaundice or breastfeeding guidelines. The testing schedule may be dynamically adapted, either automatically, or by the healthcare provider as new communications from the mother and/or output values from the Sensors 101 and 102 are received. As another example, a healthcare provider may enter the infant's age and weight at the time of discharge into the user interface provided by the Cloud Application 108. Based on this user-entered information and the Cloud Application 108 programming, the Cloud Application 108 may compute a range of desirable output values from the Breastfeeding Sensor 101, as well as threshold values for prioritizing the mother/infant pair for a higher level of attention from a healthcare provider. The Cloud Application 108 may update scheduled messaging and/or actions as a result of its updated calculations of the infant's overall health risk and/or risk of developing actionable levels of bilirubin as new output is received from the Sensors 101 and 102 and/or new communications are received from the mother. As another example, a healthcare provider may enter clinical data specific to the mother/infant pair into a Cloud Application 108 by configuring the Cloud Application 108, or causing it to be configured, to send an automated query to an EHR.

In some examples, the Cloud Application 108 may display event flags to healthcare providers via a user interface to alert them of the mother's needs or may order a list of mother/infant pairs in a displayed list according to the priority for the healthcare provider's attention. In one example, a user interface for healthcare providers displays a list of mothers who are being monitored by the Cloud Application 108 with an event flag icon in the shape of a phone displayed next to a mother's name with a color that corresponds to the urgency of the need for a direct communication with a healthcare provider. A red phone icon may indicate a need for immediate attention to the mother/infant pair, for example.

The breastfeeding period may comprise the entire first few days to weeks after the infant's release from the hospital while there is an ongoing concern regarding the infant's bilirubin levels, or it may comprise a shorter period that begins after release from the hospital in response to a concern raised at a well-baby checkup visit, for example. As used herein, the term “healthcare providers” may include medical doctors such as pediatricians or primary care physicians, physician assistants, nurses, lactation counsellors and other qualified healthcare personnel who are responsible for providing breastfeeding support to the mother and/or for monitoring the mother's and/or infant's health. The healthcare providers may be employers or contractors of the hospital where the infant was born, of a different healthcare system or physician practice that is responsible for caring for the mother/infant pair after their release from the hospital, or of a third-party private entity that provides monitoring and triaging services to hospitals or to health insurers, for example. The healthcare providers who are responsible for directly monitoring the mother/infant pairs via the Cloud Application 108 may be responsible for communicating with the mother or may simply triage incoming information from the mother/infant pair for appropriate escalation to more specialized healthcare providers. The responsibilities of a team of healthcare providers and supporting personnel may vary between hospitals according to each hospital's particular policies. Some hospitals employ fulltime lactation counsellors who serve as front-line support for mothers after discharge, while other hospitals do not have these specialized resources on staff and nurses and doctors therefore provide this type of support. Other hospitals may employ third-party agencies to provide 24-hr monitoring and first-line support to patients, thus acting as a triage stage for their own more specialized staff, for example. Independent agencies and networks of lactation counsellors who may also be trained as nurses may provide services directly to parents without any hospital or healthcare system intermediary.

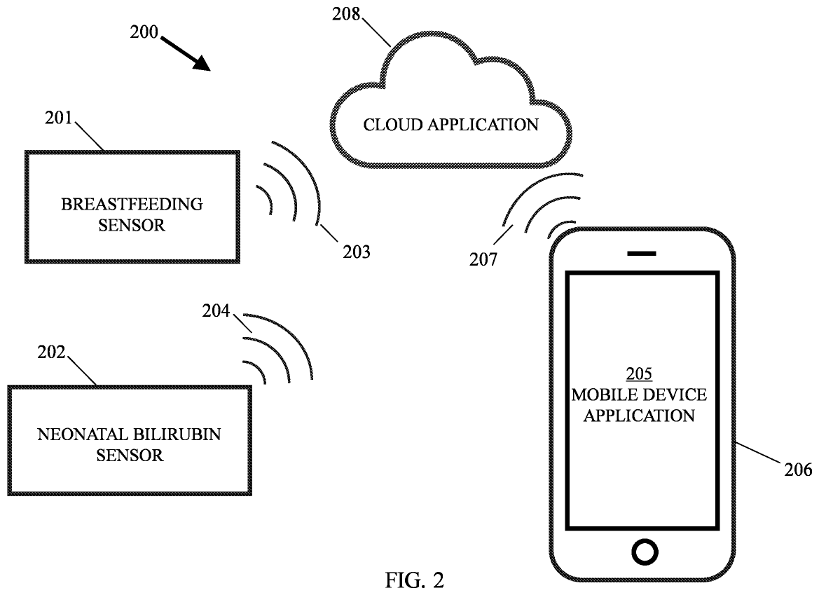

FIG. 2 illustrates a system 200 in accordance with various examples of the present disclosure for supporting breastfeeding by mothers of newborns. The system 200 comprises a Breastfeeding Sensor 201 and a Neonatal Bilirubin Sensor 202 that communicate, for example, via respective communication channels 203 and 204 directly to a Cloud Application 208 that is running on remote computer hardware. In some examples, communications over the communication channels 203 and 204 may comprise some combination of Bluetooth® communications, WiFi communications, other short-range wireless technology protocols and cellular network communications between intermediary devices. In some examples, communications over the communication channels 203 and 204 may comprise long-range wireless communications, such as a cellular communications or internet communications. In other examples, communications between the Sensors 201, 202 and the Cloud Application 208 may occur via wired connections or a combination of wired and wireless connections. A Mobile Device Application 205 may run on a Mobile Device 206 and also communicate with the Cloud Application 208, for example, via a communication channel 207. In some examples, communications over the communication channel 207 may comprise long-range wireless communications, such as a cellular communications or internet communications. In other examples, the communication channel 207 may be based on wired connections or a combination of wired and wireless connections. Those who are skilled in the art will recognize that in other examples, different communications technologies and protocols may be used.

In some examples, breastfeeding sensors and/or neonatal bilirubin sensors may comprise hardware elements that are external to a mobile device in combination with software application elements running on the mobile device. A neonatal bilirubin sensor, for example, may comprise external optical hardware components such as optical transmitters, optical receivers and/or optical lenses, or calibration cards that may be used in combination with a software application running on a smartphone to measure a parameter related to neonatal serum bilirubin levels. In some examples, a neonatal bilirubin sensor may comprise a software application running on a smartphone that leverages the internal camera of the smartphone to acquire photos of an infant's skin and an external calibration card to measure a parameter related to neonatal serum bilirubin levels. In some examples, a neonatal bilirubin sensor may comprise a software application running on a smartphone that leverages the internal camera of the smartphone to acquire photos of an external test strip that is designed for testing an infant's blood sample for bilirubin content and the software application may be configured to calculate a bilirubin level based on an analysis of the photos. In some examples, a breastfeeding sensor may comprise a nipple shield fitted with a flow-measuring apparatus that is used in combination with a software application running on a smartphone to measure a parameter related to breast milk transfer from a mother to an infant. In some examples, a plurality of external hardware components suited either for breastfeeding sensing or neonatal bilirubin sensing may be used in combination with a single software application running on a smartphone.

The Cloud Application 208 may send individualized communications to the Mobile Device 206 to be received by a mother, for example, as text messages, voice messages or emails. The Mobile Device Application 205 may provide a user interface for the mother to send communications to her healthcare providers via the Cloud Application 208.

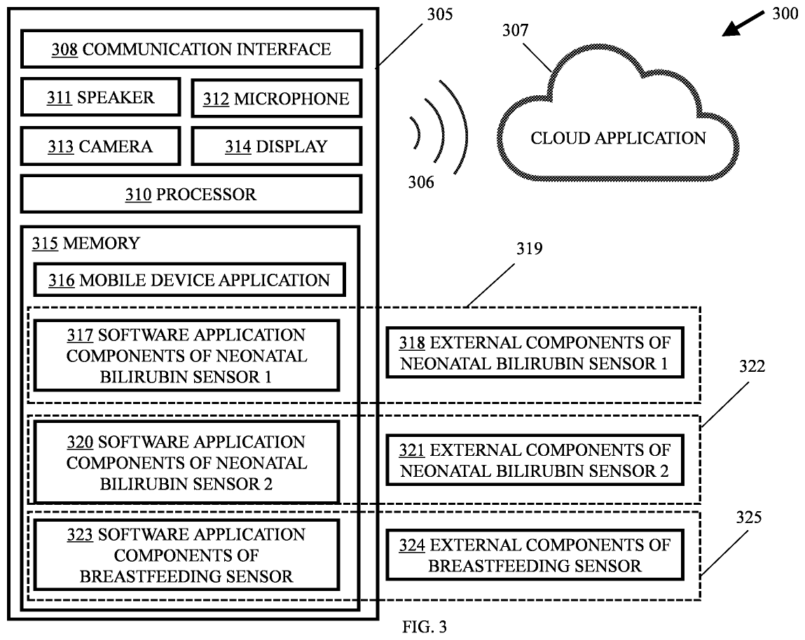

FIG. 3 illustrates a system 300 in accordance with various examples of the present disclosure for supporting breastfeeding by mothers of newborns. The system 300 comprises a first Neonatal Bilirubin Sensor 319, a second Neonatal Bilirubin Sensor 322, and a Breastfeeding Sensor 325, wherein some portions of the Sensors 319, 322, and 325 are located within a Mobile Device 305, and other portions are external to the Mobile Device 305. For example, the First Neonatal Bilirubin Sensor 319 comprises External Components 318 and Software Application Components 317, the Second Neonatal Bilirubin Sensor 322 comprises External Components 321 and Software Application components 320, and the Breastfeeding Sensor 325 comprises External Components 324 and Software Application Components 323. The Software Application Components 317, 320 and 323 are all stored in a Memory 315 of the Mobile Device 305. A Mobile Device Application 316 is also stored in Memory 315. When executed, the Mobile Device Application 316 and the Software Application Components 317, 320 and 323 run on a Processor 310 on Mobile Device 305 and may access additional internal components of the Mobile Device 305, for example, a Communication Interface 308, a Speaker 311, a Microphone 312, a Camera 313, and a Display 314. The Communication Interface 308 may provide functionality for the Mobile Device 305 to communicate via a communication channel 306 with a Cloud Application 307 that is running on computer hardware at a remote location. As in previous examples, those who are skilled in the art will recognize that there are multiple wireless communications technologies and software architectures for implementing the system in accordance with the present disclosure. For example, the Mobile Device Application 316 and the Software Application Components 317, 320 and 323 may be combined into a single software application or otherwise refactored into other suitable software architectures. The communication channel 306 may be based on a cellular connection, internet connection, combination of WiFi and cable connection or any other technology capable of long-range communication.

In some examples, the Neonatal Bilirubin Sensors 319 and 322 may be based on different sensor technologies that measure different, complementary parameters related to an infant's serum bilirubin level. For example, the First Neonatal Bilirubin Sensor 319 may comprise a noninvasive sensor that measures visual light reflectance/transmission characteristics of the infant's skin or sclera, and the Second Neonatal Bilirubin Sensor 322 may comprise an invasive sensor that measures visible changes to a test strip when a drop of blood from the infant is added to the strip. A potential advantage of including two different measurement technologies in the system 300 is that no measurement sensor is perfectly accurate, and invasive sensor technologies are typically more accurate than non-invasive blood testing methods but are not suitable for testing on a regular schedule. A schedule of more frequent testing with the noninvasive First Neonatal Bilirubin Sensor 319 may be established, with availability of the Second Neonatal Bilirubin Sensor 322 to perform an ad hoc blood test at the direction of a healthcare provider if output values from First Neonatal Bilirubin Sensor 319 are so high as to be concerning. The system 300 may be of particular value for rural mothers who may live more than a hundred miles from the nearest hospital. A timely diagnosis of severe hyperbilirubinemia is critical for appropriate treatment and avoidance of severe neurological outcomes.

In one example, the Mobile Device 305 may comprise a smartphone. The First Neonatal Bilirubin Sensor 319 may comprise a reflectance-based sensor, whose Software Application Components 317, when running on the Processor 310, send instructions to the Camera 313 to acquire digital photos of the External Components 318 which comprise a calibration card with a central hole that is placed over an infant's sternum. The Processor 310 may store the photos acquired by the Camera 313 in the Memory 315, where they may be accessed by the Software Application Components 317. The Software Application Components 317, when executed by the Processor 310, may calculate an estimated serum bilirubin level from the photos based on the pixels corresponding to the infant's skin that appear inside the central hole, while calibrating the skin color based on reference colors and grayscale values on the calibration card. The Software Application Components 317 may then store the resulting serum bilirubin level estimate in the Memory 315, where it may be accessed by the Mobile Device Application 316. Those who are skilled in the art will recognize that the acquired photos may instead be accessed and analyzed instead by the Mobile Device Application 316 directly or accessed by the Mobile Device Application 316 and then communicated to the Cloud Application 307 for analysis.

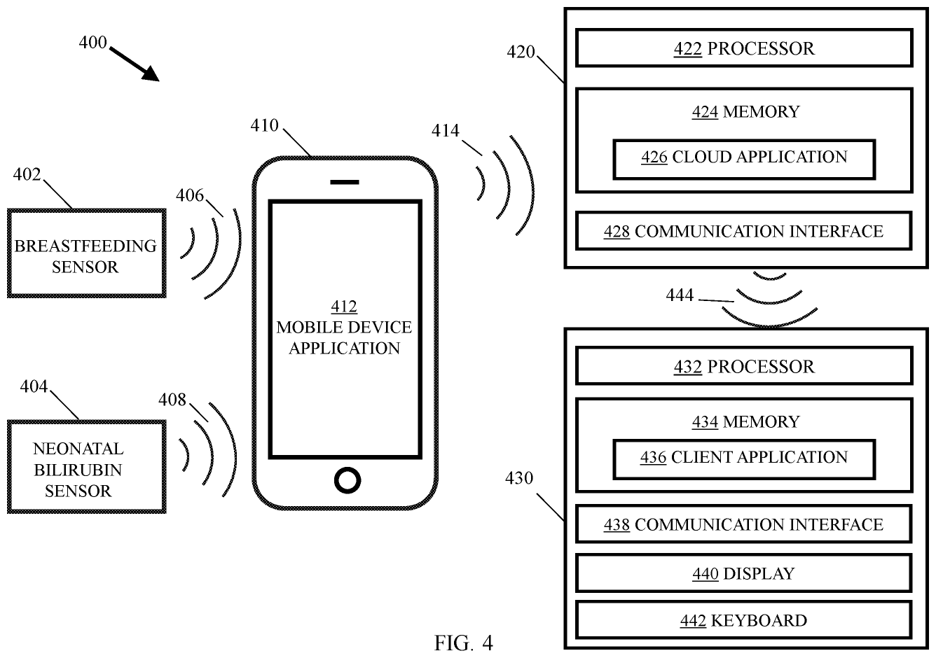

FIG. 4 illustrates a System 400 in accordance with various examples of the present disclosure for supporting breastfeeding by mothers of newborns. The System 400 comprises a Mobile Device 410 running a Mobile Device Application 412 that is programmed to receive breastmilk transfer data from a Breastfeeding Sensor 402 and bilirubin data from a Neonatal Bilirubin Sensor 404 via short-range communication channels 406, 408 respectively. The Mobile Device 410 is configured for use by a new mother in a home setting for communicating with her healthcare providers at the hospital where she gave birth to receive support with breastfeeding. The Mobile Device Application 412 is programmed to cause the Mobile Device 410 to communicate via a long-range communication channel 414 with a Cloud Server 420. A Cloud Application 426 running on the Cloud Server 420 is programmed to receive/send communications from/to the Mobile Device 410 via a Communication Interface 428 belonging to Cloud Server 420. In this example, the Cloud Application 426 is able to indirectly communicate with the Breastfeeding Sensor 402 and with the Neonatal Bilirubin Sensor 404 via the Communication Interface 428, by virtue of the short-range communication channels 406, 408 and the long-range communication channel 414. The Cloud Application 426 is stored in a Memory 424 on Cloud Server 420 and when executed, runs on a Processor 422 on the Cloud Server 420. The Cloud Server 420 comprises computing hardware that is located remotely from the mother/infant pair and may be, for example, a racked blade server in a hospital data center, i.e., in the hospital's “private cloud.” In other examples, the Cloud server 420 may be provisioned computing hardware located in third party data centers accessed by a hospital via an agreement to purchase cloud computing services from the third party, for example, Amazon Web Services. Cloud Application 426 is programmed to communicate with a Client Application 436 running on a Nurse Workstation 430. The Nurse Workstation 430 comprises computing hardware suitable for use by a healthcare professional with a user interface comprising a Display 440 and a Keyboard 442. The Client Application 436 is stored in a Memory 434 of the Nurse Workstation 430 and, when executed, runs on a Processor 432 of the Nurse Workstation 430. The Cloud Server 420 and the Nurse Workstation 430 may communicate with each other via Communication Interfaces 428, 438 and a Local Area Network (LAN) 444. The Nurse Workstation 430 may be located, for example, at a central nursing station in a newborn nursery at the hospital and configured for operation by the nursery's nursing staff. The nursery staff may be responsible for monitoring the mother/infant pair associated with the Mobile Device 410 on a 24 hr/7 day basis. The Cloud Application 426 may analyze incoming communications from the Mobile Device 410 and cause the Client Application 436 to update information about the mother/infant pair associated with the Mobile Device 410 on the Display 440. For example, the Cloud Application 426 may receive updated bilirubin data from the Mobile Device 410 that it compares to a threshold value, and, finding that the updated value exceeds the threshold, may cause the Client Application 436 to display a red flashing indicator next to the mother/infant pair's name in a list of other names on the Display 440.

Those who are skilled in the art will recognize that there are many other examples based on different choices of strategies for communicating information between the Mobile Device 410 and the Cloud Server 420, and for displaying, or otherwise communicating information about the mother/infant pair received by the Cloud Server 420 for a healthcare provider's review.

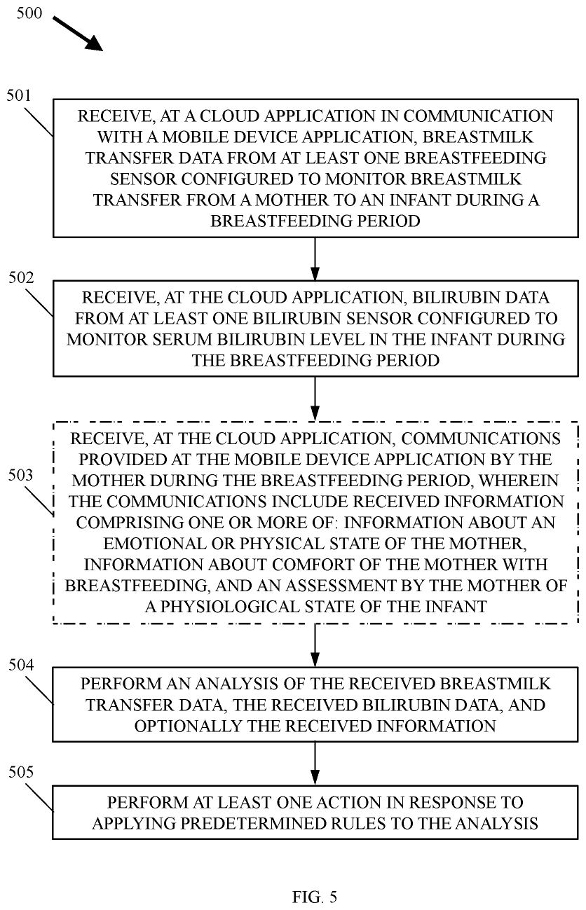

FIG. 5 illustrates an automated method 500 in accordance with various examples of the present disclosure for supporting breastfeeding by mothers of newborns during a period of breastfeeding. The method 500 may be executed, for example, by a cloud application running in a data center belonging to a hospital. The cloud application may be in communication with a mobile device application running on a mobile device possessed by a mother of an infant during a breastfeeding period, where the mobile device is remote from the cloud application. Examples of the cloud application include the Cloud Applications 108, 208, 307, and 426. Examples of the mobile device application include the Mobile Device Applications 105, 205, 316, and 412.

At 501, the cloud application receives breastmilk transfer data from at least one breastfeeding sensor configured to monitor breastmilk transfer from a mother to an infant during a breastfeeding period. Examples of the at least one breastfeeding sensor include the Breastfeeding Sensors 101, 201, 325, and 402.

At 502, the cloud application receives bilirubin data from at least one bilirubin sensor configured to monitor serum bilirubin level in the infant during the breastfeeding period. Examples of the at least one bilirubin sensor include the Neonatal Bilirubin Sensors 102, 202, 319, 322, and 404.

Optionally, at 503, the cloud application receives communications provided at the mobile device by the mother during the breastfeeding period, wherein the communications include received information comprising one or more of: information about an emotional or physical state of the mother, information about comfort of the mother with breastfeeding, and an assessment by the mother of a physiological state of the infant. In some examples, a communication received at 503 may comprise a text message sent by the mother from the mobile device in response to automated generation and transmission of a text message proactively by the cloud application to the mobile device at a scheduled time to ask how the mother is feeling, how the infant is doing, and/or if the mother has any concerns and/or wants a direct communication with a healthcare provider. In some examples, a communication received at 503 may comprise an unsolicited text message sent by the mother from the mobile device at an unscheduled time to request information and/or assistance. In some examples, the communications received at 503 are in audio format and are converted to digital files via automated speech recognition functionality.

Those who are skilled in the art will recognize that 501, 502 and 503 need not necessarily be completed in the order shown in the flowchart and that multiple instances of any of 501, 502, and 503 in any time interval within the breastfeeding period are also contemplated.

At 504, the cloud application performs an analysis of the breastmilk transfer data received at 501, the bilirubin data received at 502, and optionally, the information received at 503. In some examples, the analysis may include comparing the breastmilk transfer data received at 501 and/or the bilirubin data received at 502 to predetermined threshold values or predetermined ranges of values or to each other. In some examples, the analysis may include performing one or more calculations based on the received breastmilk transfer data and/or the received bilirubin data. In some examples, the analysis may include analyzing the contents of communications received from the mother at 503 and parsing the communications for key words or phrases that are associated with high levels of stress or would indicate an issue with the infant's state of health. In other examples, the analysis may include analyzing the mother's recorded and digitized speech for evidence of stress or panic, for example, a higher pitch to her speech and/or a more rapid speech pattern. In some examples, the analysis may include calculating a risk factor for the mother/infant pair based on any or all of such data received at 501, 502 and 503. In some examples, the risk factor may be calculated using clinical data associated with the mother and/or the infant entered by at least one healthcare provider, for example, before the mother has been discharged from the hospital. In some examples, the risk factor may be calculated using AI algorithms to compare to a large database of relevant data and known outcomes. For example, by comparing to a large database of relevant data and known outcomes, a lower risk for a particular bilirubin level may be calculated for infants who are receiving large volumes of milk through successful breastfeeding versus a higher risk for infants with the same measured bilirubin level and less successful milk transfer from the mother. In some examples, the individualized risk factor is calculated using a neural network model trained with reference data.

At 505, the cloud application performs at least one action in response to applying predetermined rules to the analysis performed at 504. As an example, where the analysis at 504 comprises comparing the received breastmilk transfer data to a predetermined range of values, a predetermined rule may be that the cloud application is to set an event flag in response to determining that the received breastmilk transfer data is not within the predetermined range of values. In this case, the action performed at 505 is that the cloud application sets an event flag to alert a healthcare provider that the mother/infant pair needs their attention. In some examples, the event flag comprises a visible marker on a computer display, such as the Display 440, that is in communication with the cloud application and being monitored by the healthcare provider. In alternative examples, the event flag comprises an audible alarm emitted by a device that is in communication with the cloud application. In other examples, the event flag comprises a text message phone call with an audio format message sent by the cloud application to the healthcare provider.

In another example, the cloud application may determine by following the predetermined rules that no event flag needs to be created, in which case the at least one action performed at 505 may comprise refraining from creating an event flag and continuing to monitor for subsequent breastmilk transfer data received at 501, subsequent bilirubin data, and/or subsequent communications received from the mother at 503. In some examples, the at least one action may comprise providing individualized content to the mother via the mobile device application based, for example, on an individualized risk factor calculated at 504.

In an exemplary workflow, the method 500 begins when the mother arrives home with her 3 day-old infant after leaving the hospital and repeats over the following days with different numbers of repeated instances of 501, 502 and 503 in varying order. Prior to release from the hospital, the infant had already been tested using TSB, and the test result indicated that the infant was in the low intermediate risk zone. However, the mother is of Sub-Saharan African heritage and is aware that her baby is therefore at higher risk of having a glucose-6-phosphate dehydrogenase (G6PD) deficiency, and therefore at higher risk for developing severe hyperbilirubinemia. At the hospital, the mother benefited from breastfeeding support and the mother/infant pair were considered to have achieved a pattern of successful latching and feeding. The hospital provided the mother with a smartphone and external sensor components for collecting breastmilk transfer data and bilirubin data for the infant, and for communicating with the hospital staff. The mother breastfeeds her infant throughout their first day and night at home whenever the infant seems hungry but on the second day at home, she is uncertain whether she is producing enough milk because the infant has begun to sleep for shorter periods and seems fussier than when they were at the hospital. In addition, the mother is alone during the day with the baby and a toddler and is finding less time available with the older child due to the increased frequency of breastfeeding. She is beginning to feel stressed and guilty and wants to be assured that the new infant is receiving adequate nutrition and that his bilirubin levels are not rising more than normal as a result of a breastfeeding issue that she cannot identify. At an occurrence of 503, the mother sends a text message using the smartphone she was provided at the hospital, for example as shown in 106, 206, 305, or 410 with message content that she has concerns her baby is not receiving enough milk from breastfeeding. The smartphone communicates the text message to a cloud application that is programmed to execute the method 500, for example, the cloud application 108, 208, 307, or 426. At 503, the cloud application receives the text message sent by the mother and sends a reply text to the mother's smartphone, with scripted questions designed to elicit more information about her emotional state, comfort with breastfeeding and the infant's state of health. Output breastmilk transfer data from a breastfeeding sensor, for example, the breastfeeding sensor 101, 201, 325, or 402, has been received for all previous feedings at multiple prior instances of 501. One prior measurement of bilirubin levels was received by the cloud application from a neonatal bilirubin sensor, for example, the bilirubin sensor 102, 202, 319, 322, or 404, during the previous day (Day 1 at home) in a single instance of 502. The cloud application therefore sends a text message to the mother asking her to perform a new bilirubin measurement and then, via a series of informative text messages, guides her through the process of using the neonatal bilirubin sensor. After the mother successfully follows the instructions in the series of text messages and a bilirubin measurement has been made, the cloud application receives the bilirubin data from the neonatal bilirubin sensor in a new instance of 502. The cloud application then performs an analysis 504 which includes calculating a risk factor based on the infant's age, weight and bilirubin level at the hospital that had been entered by a healthcare provider for the mother/infant pair prior to discharge, as well as output data from the instances of 501 and 502, and any communications received from the mother at 503 to-date. Based on this risk factor and a set of predetermined threshold values for the risk factor, the cloud application computes that it is not necessary to create an event flag to alert a healthcare provider to the mother/infant's situation and instead, at 505, performs the actions of sending a scripted text message to the mother reassuring her and asking her if she would like to see an educational video about breastfeeding, as well as offering her the option to request a phone call from a healthcare provider, and continuing to monitor for additional incoming text messages from the mother's smartphone and/or incoming data from the breastfeeding or bilirubin sensors.

The automated method 500 functions to analyze information about the mother/infant pair at multiple timepoints during a breastfeeding period, to provide individualized communications to the mother to help her overcome breastfeeding issues and encourage her to continue with breastfeeding, and to identify when a mother/infant pair would benefit from increased attention and/or direct communication with a healthcare provider.

The automated method 500 may be used to monitor a plurality of mother/infant pairs simultaneously. For example, the method 500 may further comprise receiving additional communications provided at additional instances of the mobile device application by additional mothers of additional infants, the additional communications including additional received information; receiving additional breastmilk transfer data from additional breastfeeding sensors configured to monitor breastmilk transfer from the additional mothers; receiving additional bilirubin data from additional bilirubin sensors configured to monitor serum bilirubin level in the additional infants; performing additional analyses of the additional information, the additional breastmilk transfer data, and the additional bilirubin data; and performing additional actions in response to applying the predetermined rules to the additional analyses.

The method 500 may be implemented, for example, within any one of the systems 100, 200, 300, and 400.

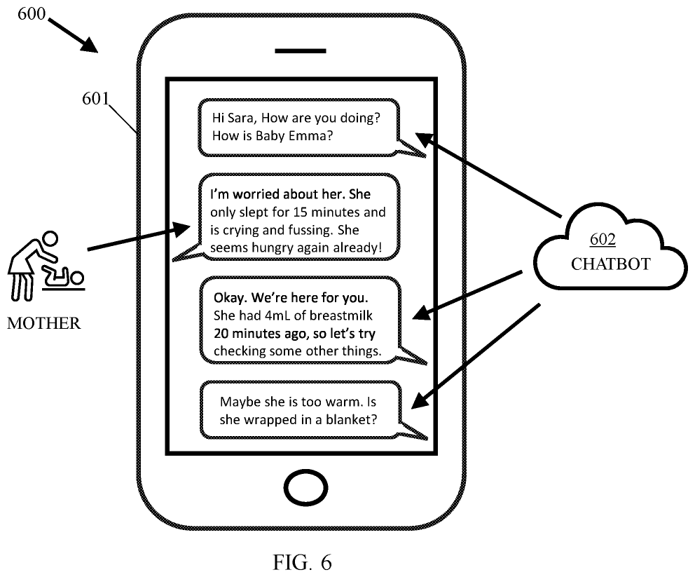

FIG. 6 illustrates an exemplary communication 600 between a mother and chatbot functionality of a cloud application, “the Chatbot,” 602 during a breastfeeding period in accordance with various examples of the present disclosure for supporting breastfeeding by mothers of newborns during a period of breastfeeding based on deploying a Smartphone 601 and sensors (not shown) to collect breastmilk transfer data and bilirubin data in the home setting for the mother/infant pair. In this exemplary communication, the Chatbot 602 proactively sends a text to the mother's Smartphone 601 to ask how she is doing. Using the Smartphone 601, the mother sends a reply text message that she is concerned about the infant's fussing. The Chatbot 602 parses and interprets the text message using its natural language processing capabilities, selects an appropriate scripted response for reassuring the mother that it can help her, sends the response as a text message to the mother's Smartphone 601, calculates a risk factor by comparing the most recently received breastmilk transfer data to a threshold value, selects an appropriate response corresponding to the calculated risk factor, for example, a scripted response that reassures the mother that the infant received sufficient breastmilk at the last feeding, sends a text message with the response to the mother's Smartphone 601, selects an appropriate scripted message asking the mother to check for other possible sources of the fussing, and then sends the text message to the mother's Smartphone 601.

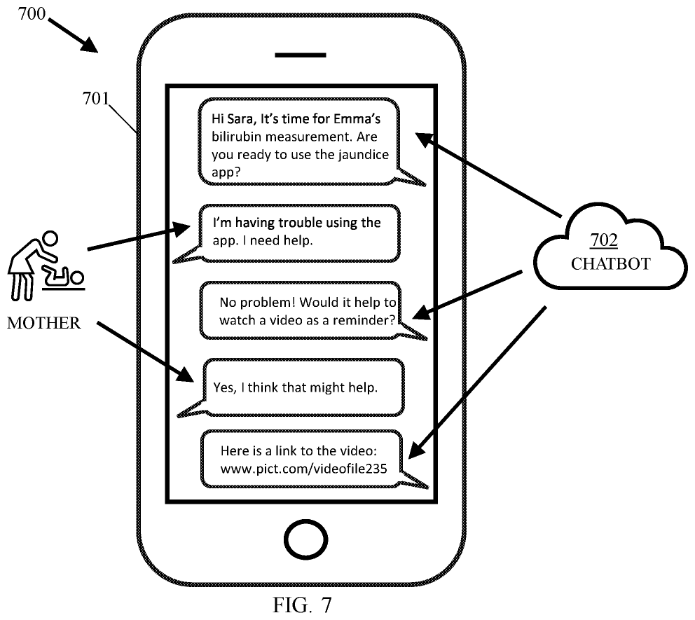

FIG. 7 illustrates an additional exemplary communication 700 between a mother and the chatbot functionality of a cloud application, “the Chatbot,” 702 during a breastfeeding period in accordance with various examples of the present disclosure for supporting breastfeeding by mothers of newborns during a period of breastfeeding based on deploying a Smartphone 701 and sensors to collect breastmilk transfer data and bilirubin data in the home setting for the mother/infant pair (not shown). In this exemplary communication, the Chatbot 702 sends the mother's Smartphone 701 a text message at a scheduled time with a reminder to notify her that it is time for her to make a bilirubin measurement. Using the Smartphone 701, the mother sends a text message saying that she is having difficulty using the neonatal bilirubin sensor. The Chatbot 702 parses and interprets the text message, makes a decision that the mother is indicating that she is having difficulty using the neonatal bilirubin sensor, selects an appropriate scripted response message that offers to provide a helpful training video, and sends a text message with the response message to the mother's Smartphone 701. Using the Smartphone 701, the mother sends a reply text message indicating that she would like to watch the video. The Chatbot 702 selects an appropriate choice from a list of hyperlinks to video files available on the internet and sends a text message to the mother's Smartphone 701 with the hyperlink.

Although the disclosed subject matter has been described and illustrated with respect to examples thereof, it should be understood by those skilled in the art that features of the disclosed examples can be combined, rearranged, etc., to produce additional examples within the scope of the invention, and that various other changes, omissions, and additions may be made therein and thereto, without parting from the spirit and scope of the present invention.

What is claimed is:

1. An automated method for remotely supporting breastfeeding mothers, the method comprising:
receiving, at a cloud application in communication with a mobile device application, breastmilk transfer data from at least one breastfeeding sensor configured to monitor breastmilk transfer from a mother to an infant during a breastfeeding period;
receiving, at the cloud application, bilirubin data from at least one bilirubin sensor configured to monitor serum bilirubin level in the infant during the breastfeeding period;
performing an analysis of the received breastmilk transfer data and the received bilirubin data; and
performing at least one action in response to applying predetermined rules to the analysis.

2. The method as claimed in claim 1, further comprising:
receiving, at the cloud application, communications provided at the mobile device application by the mother during the breastfeeding period, wherein the communications include received information comprising one or more of: information about an emotional or physical state of the mother, information about comfort of the mother with breastfeeding, and an assessment by the mother of a physiological state of the infant, and
wherein performing the analysis comprises analyzing the received information.

3. The method as claimed in claim 1, wherein the at least one action comprises creating an event flag in association with the mother to trigger attention from at least one healthcare provider.

4. The method as claimed in claim 3, wherein creating the event flag comprises one or more of:
displaying a visual indicator on a display associated with the cloud application; and
initiating a digital communication between the cloud application and the at least one healthcare provider.

5. The method as claimed in claim 2, wherein the at least one action comprises:
refraining from creating an event flag in association with mother; and
monitoring for one or more of (i) subsequent breastmilk transfer data from the at least one breastfeeding sensor; (ii) subsequent bilirubin data from the at least one bilirubin sensor;
and (iii) subsequent communications provided at the mobile device application.

6. The method as claimed in claim 1, wherein the analysis comprises comparing the received breastmilk transfer data and the received bilirubin data to predetermined threshold values and/or predetermined ranges of values and/or to each other.

7. The method as claimed in claim 2, wherein the analysis comprises calculating an individualized risk factor associated with the mother and/or the infant using the received information, the received breastmilk transfer data, and the received bilirubin data.

8. The method as claimed in claim 7, wherein the individualized risk factor is calculated using one or more of:
clinical data associated with the mother and/or the infant entered by at least one healthcare provider; and
a neural network model trained with reference data.

9. The method as claimed in claim 7, wherein the at least one action comprises providing individualized content to the mother via the mobile device application based on the individualized risk factor.

10. The method as claimed in claim 2, wherein the communications provided by the mother comprise text communications, the method further comprising:
extracting the received information using a natural language processing algorithm configured to parse the text communications for key words and/or phrases associated with stress and/or health of the mother and/or the infant.

11. The method as claimed in claim 2, further comprising:
receiving, at the cloud application, additional communications provided at additional instances of the mobile device application by additional mothers of additional infants, the additional communications including additional received information;
receiving, at the cloud application, additional breastmilk transfer data from additional breastfeeding sensors configured to monitor breastmilk transfer from the additional mothers;
receiving, at the cloud application, additional bilirubin data from additional bilirubin sensors configured to monitor serum bilirubin level in the additional infants;
performing additional analyses of the additional information, the additional breastmilk transfer data, and the additional bilirubin data; and
performing additional actions in response to applying the predetermined rules to the additional analyses.

12. A system comprising:
a processor;
a communication interface configured to communicate with at least one instance of a mobile device application, with at least one breastfeeding sensor configured to monitor breastmilk transfer from a mother to an infant during a breastfeeding period, and with at least one bilirubin sensor configured to monitor serum bilirubin level in the infant during the breastfeeding period;
a computer-readable medium storing computer-executable instructions for a cloud application which, when executed by the processor, cause the cloud application:
to perform an analysis of breastmilk transfer data received from the at least one breastfeeding sensor during the breastfeeding period and bilirubin data received from the at least one bilirubin sensor during the breastfeeding period; and
to perform at least one action in response to applying predetermined rules to the analysis.

13. The system as claimed in claim 12, wherein the analysis further comprises analyzing information received in communications provided at the mobile device application by the mother during the breastfeeding period, the received information comprising one or more of:
information about an emotional state of the mother, information about comfort of the mother with breastfeeding, and an assessment by the mother of a physiological state of the infant.

14. The system as claimed in claim 12, wherein the at least one action comprises creating an event flag in association with the mother to trigger attention from at least one healthcare provider, the event flag comprising one or more of:
a visual indicator displayed on a display of the electronic device; and
a communication initiated with the at least one healthcare provider.

15. The system as claimed in claim 12, wherein the analysis comprises comparing the received breastmilk transfer data and the received bilirubin data to predetermined threshold values and/or to predetermined ranges of values and/or to each other.

16. The system as claimed in claim 13, wherein the analysis comprises calculating an individualized risk factor associated with the mother and/or the infant using the received information, the received breastmilk transfer data, and the received bilirubin data.

17. The system as claimed in claim 16, wherein the individualized risk factor is calculated using one or more of:
clinical data associated with the mother and/or the infant entered by at least one healthcare provider; and
a neural network model trained with reference data.

18. The system as claimed in claim 15, wherein the at least one action comprises transmitting to the mobile device application, via the communication interface, content based on the individualized risk factor.

19. The system as claimed in claim 13, wherein the communications provided by the mother comprise text communications, and wherein execution of the computer-executable instructions further causes the cloud application:
to extract the received information using a natural language processing algorithm configured to parse the text communications for key words and/or phrases associated with stress and/or health of the mother and/or the infant.

20. The system as claimed in claim 12, wherein execution of the computer-executable instructions further causes the cloud application:
to perform additional analyses of: (i) additional breastmilk transfer data from additional breastfeeding sensors configured to monitor breastmilk transfer from additional mothers of additional infants; (ii) additional bilirubin data from additional bilirubin sensors configured to monitor serum bilirubin level in the additional infants; and (iii) additional information included in additional communications provided by the additional mothers at additional instances of the mobile device application; and
to perform additional actions in response to applying the predetermined rules to the additional analyses.
